(19) United States
(12) Patent Application Publication
(10) Pub. No.: US 2004/0198957 A1
(43) Pub. Date: Oct. 7, 2004

(54) Method for removing endotoxins from protein solutions

(76) Inventors: Inna Way, Pearland, TX (US); Simon Hoffenberg, Hartsdale, NY (US); Taeha An, Houston, TX (US); Jeffrey Su, San Diego, CA (US)

Correspondence Address: Wendell Ray Guffey, Tanox Inc, Suite 110, 10301 Stella Link, Houston, TX, 77025-5497, US

(21) Appl. No.: 10/488,161

(22) PCT Filed: Aug. 23, 2002
(86) PCT No.: PCT/US02/27255

Related U.S. Application Data
(60) Provisional application No. 60/315,197, filed on Aug. 27, 2001.

(51) Int. Cl. C07K 16/18

(52) U.S. Cl. 530/387.1; 530/413

(57) ABSTRACT

A method for removing endotoxin contaminants from protein solutions using hydrophobic charge induction chromatography sorbents. The methods comprise adjusting the ph of a protein solution to a pH of from about 8.0 to about 9.0, binding the protein to a hydrophobic charge induction chromatography sorbent; and eluting the protein from the sorbent using an elution buffer having a pH of from about 3.0 about 5.0. The method is particularly useful for removing endotoxin contaminants from antibody compositions.

### CROSS REFERENCE TO RELATED APPLICATIONS

This application claims priority to U.S. Provisional Application Serial No. 60/315,197, filed Aug. 27, 2001, the disclosure of which is incorporated herein by this reference.

### BACKGROUND OF THE INVENTION

1. Field of the Invention

This invention relates generally to endotoxins and particularly to methods for removing endotoxin contaminants from protein solutions and to the protein compositions produced by such methods.

2. Description of the Prior Art

Endotoxins, the lipopolysaccharide components of gram-negative bacterial cell walls, are a significant contaminant in proteins made using biotechnology production methods. When bacteria are used to produce proteins, endotoxins are released into a protein solution from the bacteria. When mammalian cells are used to produce proteins, absolute sterility during the protein production process is required to avoid bacterial and therefore endotoxin contamination. However, absolute sterility is difficult if not impossible. Endotoxin contamination often results from contamination or raw materials, buffers, water used as a solvent, growth media used for cell culture, and devices used for purification.

Bacterial endotoxins are usually harmful and potentially fatal (Grandics, Pharmaceutical Technology, Apr. 27-34 (2000)). Their biological effects are triggered at concentrations as little as a few nanograms per kilogram body weight. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as in case of monoclonal antibodies, even trace amounts of harmful and dangerous endotoxin must be removed.

Common purification methods for monoclonal antibodies, that include protein A and protein G affinity chromatography, ion-exchange, hydrophobic interaction, and hydroxyapatite resins, have been employed for endotoxin clearance with different success (Bischoff et al, Biochemistry 30:3464-3472 (1991); Neidhardt et al., J. Chromatogr. 590:255-261 (1992); Kang and Luo, J. Chromatogr. 809:13-20 (1998); Fiske et al., J. Chromatogr. B Biomed. Sci. Appl. 753 (2):269-278 (2001); Wilson et al., J. Biotechnol. 88 (1), 67-75 (2001)). In some cases, even special endotoxin-selective adsorbents, such as polymyxin B, histidine and poly-lysine, did not achieve acceptable clearance rates for endotoxin (Liu et al, Clinical Biochem. 30:455-463 (1997)). They are particularly ineffective if a contaminant binds strongly to the target protein.

None of the above cited methods can be relied upon to remove endotoxins and ensure the safety of the proteins produced using biotechnology production methods. There is, therefore, a need for new and improved methods for producing proteins that are substantially free of endotoxin contaminants.

### SUMMARY OF THE INVENTION

It is, therefore, an object of the invention to provide methods for removing endotoxin contaminants from protein solutions.

It is another object of the invention to provide protein compositions that are substantially free from endotoxin contaminants.

It is a further object of the present invention to provide protein compositions that can be administered as drugs to humans and animals.

These and other objects are achieved using novel and efficient methods for removing endotoxin contaminants from protein solutions. The endotoxins are removed by adjusting the pH of a protein solution to a pH of from about 8.0 to about 9.0, binding the protein to a hydrophobic charge induction chromatography sorbent; and eluting the protein from the sorbent using an elution buffer having a pH of from about 3.0 to about 5.0. Typically, the protein solution is obtained from the recombinant production of proteins using recombinant bacteria fermentation or mammalian cell culture. The hydrophobic charge induction chromatography sorbent comprises a cellulose matrix linked to 4-Mercapto-Ethyl-Pyridine (4-MEP) ligand or its analogs. The pH of the protein solution and the buffers is adjusted using well known methods and compositions. These methods are useful for preparing protein compositions substantially free from endotoxin contaminants, including antibody and antibody fragment protein compositions.

Other and further objects, features and advantages of the present invention will be readily apparent to those skilled in the art.

### BRIEF DESCRIPTION OF THE DRAWINGS

FIG. 1 shows elution profiles of a monoclonal antibody (mAb1) on a hydrophobic charge induction chromatography sorbent column.

FIG. 2 shows elution profiles of a monoclonal antibody (mAb2) on a hydrophobic charge induction chromatography sorbent column.

FIG. 3 shows elution profiles of two monoclonal antibodies (mAb1 (A) and mAb2 (B)) on a Phenyl Sepharose 6 Fast Flow 1 ml HiTrap column.

### DETAILED DESCRIPTION OF THE INETION

The term “substantially free of endotoxin contaminants” as used herein means that the concentration of endotoxins in a protein composition is equal to or less than the amount permitted by the Food & Drug Administration (“FDA”) or an equivalent agency in protein compositions to be administered to humans or other animals as drugs. Preferably, the endotoxin concentration is equal to or less than 5 endotoxin units (EU) per dose per kilogram body weight when administered intravenously in a one hour period.

In one aspect, the present invention provides methods for removing endotoxins from protein solutions. The methods comprise adjusting the pH of a protein solution to a pH of from about 7.5 to about 10.5, preferably from about 8.0 to about 9.0, binding the protein to a hydrophobic charge induction chromatography sorbent, and eluting the protein from the sorbent using an elution buffer having a pH of from about 2.5 to about 5.0, preferably from about 3.0 to about 4.0. The methods provide a rapid, one-step process for removing endotoxin contaminants from protein solutions, particularly antibody compositions comprising antibodies or antibody fragments such as the human IgG Fc fragment.

In a preferred embodiment, the methods further comprise an additional step before eluting the protein from the sorbent. In this method, the protein bound to the hydrophobic charge induction chromatography sorbent is washed using a buffer having a pH of from about 7.0 to about 7.5 before elution using the lower pH buffer. This washing step removes some undesirable materials from the column before it is eluted with the lower pH buffer to recover the desired protein.

When the protein is an antibody or antibody fragment, the antibody or fragment can be a selected from any class of immunoglobin including IgA, IgD, IgE, IgG, or IgM. The fragment can be a Fc or Fab fragment from any class of antibody. Preferably, the fragment is a human immunoglobulin Fc fragment, most preferably an IgG Fc fragment, most preferably an IgG fragment of the IgG4(γ4) subclass.

The protein solution useful in the present invention can be any protein solution thought to contain endotoxin contaminants. The protein solution can be one produced naturally from any protein production process, particularly recombinant protein production processes using bacteria. Alternatively, the protein solution can be one produced using mammalian, insect, yeast, or other cell culture systems. Preferably, the protein solution is the result of a recombinant protein production and purification process that used recombinant bacteria to produce the protein.

The hydrophobic charge induction chromatography sorbent comprises a cellulose matrix linked to 4-Mercapto-Ethyl-Pyridine (4-MEP) ligand or its analogs. 4-MEP analogs useful in the present invention include, but are not limited to, compounds having the structure 4-A-B-C, where A is an amino or hydroxyl group; B is a hydrophobic moiety, preferably a linear or branched hydrocarbon having from 1 to 8 carbon atoms, and C is pyridine.

The cellulose beads provide the sorbent with high porosity, chemical stability, and low non-specific interaction with proteins. The ligand permits the sorbent to interact with the protein through a mild hydrophobic interaction without the addition of lyotropic or other salts.

The cellulose bead size depends upon the protein solution to be treated. Preferably, the cellulose beads have a size of about 80-100 μm. This size permits the sorbent to have an acceptable capacity while maintaining an acceptable flow rate, particularly for antibodies and fragments thereof.

A 4-MEP ligand is particularly useful for the purification of antibody protein compositions because of the ligand's ability to interact with antibodies and fragments thereof. The ligand contains a hydrophobic tail and an ionizable headgroup. At physiological pH, the aromatic pyridine ring is uncharged and hydrophobic. Also, the aliphatic spacer arm contributes to binding of proteins. The ligand is highly selective and has a high capacity for antibodies. Its pKa of about 4.8 makes the ligand particularly suitable for interaction with antibodies. Antibody binding is further enhanced by interaction with the thioether group. Both ligand structure and ligand density are designed to provide effective binding in the absence of lyotropic or other salts.

Protein desorption is based on the charge repulsion that occurs when the pH is reduced. When pH is adjusted to values below about 4.8, preferably from about 3.5 to 4.5, most preferably to about 4.0, the ligand takes on a distinct positive charge. Under such conditions, proteins also carry a positive charge. Electrostatic repulsion is induced and the protein is desorbed. Elution buffers useful for adjusting the pH are well known to skilled artisans. Preferably, the elution buffer is a 50 mM citric acid buffer having a pH in the required range.

In contrast to traditional hydrophobic interaction chromatography, hydrophobic charge induction chromatography uses pH rather than salt concentration to control the process. Protein elution is conducted at low ionic strength thus eliminating the need for extensive diafiltration in applications where ion exchange chromatography will follow capture. Compared to chromatography on Protein A sorbents, elution from hydrophobic charge induction chromatography sorbents is achieved under relatively mild conditions (pH 4.0). The mild conditions help to reduce aggregate formation and maintain protein activity. Compared to prior methods, the present invention is particularly useful for removing endotoxin contaminants to very low levels, i.e., preparing protein compositions that are substantially free of endotoxin contaminants.

Hydrophobic charge induction chromatography absorbents useful in the present invention can be obtained from Ciphergen Biosystems, Inc., 6611 Dumbarton Circle, Fremont, Calif. 94555 under the trademark BioSepra® MEP HyperCel™ Sorbent or from other vendors such as Invitrogen Corp (Rockville, Md., USA). These sorbents are known to be useful for capture and purification of monoclonal and polyclonal antibodies (Guerrier et al, In: Subramanian, G. (Ed.), European Conference on Antibodies Production and Purification, Paris, France, Oct. 27-29, 1-9(1999)). Absorption of antibodies is based on molecular recognition of the ionizable ligand and mild hydrophobic interaction and is achieved without the addition of salts. Desorption is based on ion exchange repulsion by reducing the pH of the mobile phase (Boschetti, Genetic Engineering News, 20 (13):1-4 (2000)). While this sorbent has a few advantages over traditional Protein A and G chromatography, such as ligand stability during 1 M sodium hydroxide cleaning and more gentle pH-controlled elution, it has a nonspecific affinity for albumin. This requires an additional washing step with sodium caprylate, when working with serum-containing cell culture supernatant (Schwarz, In: Subramanian, G. (Ed.), European Conference on Antibodies Production and Purification. Paris, France, Oct. 27-29, 18-21 (1999)).

In one embodiment, the protein solutions are pre-purified using known techniques before using the present invention to remove the endotoxin contaminants. Preferred techniques for pre-purification include affinity chromatography, ion-exchange chromatography, hydrophobic interaction chromatography, and hydroxyapatite resin chromatography. Preferably, the pre-purification uses Protein A Hydroxyapatite affinity chromatography or Prosep A affinity chromatography.

Solutions, compounds, and methods for adjusting the pH in the present invention are well known to skilled artisans.

The methods of the present invention are useful for removing endotoxins from any protein solution but are particularly suitable for removing endotoxins from antibodies and fragments thereof.

In another aspect, the present invention provides protein compositions that are substantially free from endotoxin contaminants. Such compositions are prepared using the methods of the present invention. The protein compositions of the present invention are useful as drugs for treating various diseases in humans and other animals. The drugs containing protein compositions substantially free of endotoxin contaminants can be administered to the patient without causing the adverse side effects characteristic of endotoxins, e.g., fever, flu-like-symptoms, headache, vomiting, and diarrhea.

This invention can be further illustrated by the following examples of preferred embodiments thereof, although it will be understood that these examples are included merely for purposes of illustration and are not intended to limit the scope of the invention unless otherwise specifically indicated.

### EXAMPLES

### Materials and Methods

All chromatographic procedures were performed on AKTA explorer 10 S HPLC system from Amersham-Pharmacia (Piscataway, N.J., USA). The system was depyrogenated with 1 N sodium hydroxide each time before use. Prosep A chromatography media was from BioProcessing (Consett, Co. Durham, UK). MEP HYPERCEL™ media was from Life Technologies, Inc. (Rockville, Md., USA). Phenyl Sepharose 6 Fast Flow (low sub) HiTrap column was from Amersham-Pharmacia. Lumulus amebocyte lysate (LAL) assay kit was from Charles River Laboratories (Charleston, S.C., USA). 15-liter cell culture bioreactor was from Applikon (Foster City, Calif., USA). M6 Tangential Filtration System was from Millipore (Bedford, Mass., USA). All buffers were made with analytical grade reagents prepared with Sterile Water for Injections (SWI) (axter Healthcare, Miami, Fla., USA), and sterile filtered using Millipore Durapore 0.22 μm membranes (Millipore, Bedford, Mass., USA). Sterile, disposable plasticware was used to prevent endotoxin contamination. All glassware was depyrogenated by heating at 200° C. for at least 4 h. Endotoxin from E. coli 055:B5 (Charles River Laboratories) was used to spike antibody solutions.

### Example 1

### Antibody Production

Chimeric mouse-human anti-CD86 monoclonal antibody (antibody 1, mAb1) and anti-CD4 monoclonal antibody (antibody 2, mAb2), both containing human IgG4 Fc fragment and the light chains of the kappa type were produced in NS0 transfectoma cells. Cells were cultured in Iscove's modified Dulbecco's medium (GIBCO, Grand Island, N.Y., USA) supplemented with insulin (Sigma, St. Louis, Mo., USA) at 5 mg/L, human transferrin (Sigma) at 5 mg/L, and 2% fetal calf serum (GIBCO). Hypoxanthine (Sigma) at 1 μg/ml, xanthine (Sigma) at 250 μg/ml, and mycophenolic acid (Sigma) at 15 μg/ml were used as selection reagents. Cells were propagated in 2-liter spinner flasks to the density of 2×106/ml. These cultures were used for inoculation of 15-liter bioreactors (Applikon). After 12 days, cell culture supernatants were harvested.

Clarification and concentration were performed by tangential flow filtration on M6 Tangential Filtration System (Millipore). For 10-fold concentration three Pelicon filter cassettes with a nominal molecular weight cut off of 30000 (Millipore) were used. Concentrated cell culture supematants, supplemented with 0.02% of sodium azide were stored at 4° C. until purification.

### Example 2

### Antibody Purification

Antibodies were purified by affinity chromatography on XK 26/30 column (Amersham-Pharmacia) packed with 100 ml of Prosep A (BioProcessing). Before each use the column was cleaned with 6 M guanidine hydrochloride and washed with endotoxin-free SWI. Cell culture supernatants were filtered through ZapCap-S cellulose acetate bottle-top 0.22 μm filters (VWR Scientific, Bridgeport, N.J., USA). The loading buffer was Dulbecco's Phosphate-Buffered Saline (PBS), pH 7.2 without calcium chloride and without magnesium chloride (Life Technologies). The flow rate was 120 cm/h. The column was first equilibrated with 10 column volumes (CV) of loading buffer. Next, the culture supernatant was applied to the column and the column was washed with loading buffer until the UV detector signal had reached the baseline. Bound proteins were eluted with 50 mM citric acid, pH 3.0.

Eluted antibody was immediately neutralized with 1 M Tris-HCl, pH 9.0. Purified antibody was dialyzed against PBS and concentrated in a 400 ml Amicon filtration unit (Fisher Scientific, Pittsburg, Pa., USA ) with YM30 regenerated cellulose membrane (Millipore). The antibody concentration in the final preparations was measured at 280 nm and calculated using an extinction coefficient of 1.4 ml-1 cm-1. All purification experiments were carried out at room temperature.

### Example 3

### Endotoxin Removal on MEP HYPERCEL™

### Small-Scale Purification

MEP HYPERCEL™ chromatography was carried out with a C 10/10 column containing 6.28 ml MEP HYPERCEL™. The flow rate was kept constant at 76 cm/h. The column was cleaned with 6 M guanidine hydrochloride and washed with endotoxin-free SWI before each use. The gel was sanitized with 1 N sodium hydroxide for 1 h, washed with SWI until neutrality and equilibrated with 10 CV of PBS. The sample was filtered through cellulose acetate 0.22 μm filter (Corning Inc., Corning, N.Y., USA) and applied to the column. Antibody concentration in the load was 2 mg/ml. Next, the column was washed with 10-15 CV of PBS. Bound protein was eluted with 50 mM sodium citrate, pH 4.0 and pH 3.0, and pH was adjusted to 7.5 with 1 M Tris-HCl, pH 9.0. 2 ml fractions were collected for endotoxin analysis.

Scaled up purification. For purification of antibodies on a gram-scale MEP HYPERCEL™ chromatography was performed with 250 ml of MEP HYPERCEL™ packed in an XK 50/20 column with an adapter. The flow rate was 30 cm/h except for loading when it was decreased to 20 cm/h. Antibody concentration in the load was 5 mg/ml. Cleaning the column with 6 M guanidine hydrochloride and 1 N sodium hydroxide for 1 h, equilibration with PBS, elution with 50 mM sodium citrate, pH 3.0, was similar to the small-scale procedure. After loading, the column was washed with 4 CV of PBS. Eluted protein was collected in a single container and neutralized to pH 7.5 with 1 M Tris-HCI, pH 9.0.

### Example 4

### Chromatography on Phenyl Sepharose 6 Fast Flow

Phenyl Sepharose 6 Fast Flow (low sub) 1 ml HiTrap column was purchased from Amersham-Pharmacia. The column was equilibrated with 20 mM Tris-HCl, pH 7.7, containing 1 M ammonium sulfate. The flow rate was 1 ml/min. 500 μl of 2 mg/ml antibody solution, supplemented with ammonium sulfate to the final concentration of 1 M, were applied to the column. The column was washed with 20 mM Tris-HCl, pH 7.7, containing 1 M ammonium sulfate. Bound antibody was eluted with a negative gradient of 1 M ammonium sulfate in 20 mM Tris-HCl, pH 7.7.

### Example 5

### Endotoxin Assay

Endotoxin units were measured using LAL ENDOCHROME™ kit (Charles River Laboratories) according to manufacturer's instruction. Briefly, 50 μl of serially diluted samples and control standard endotoxin were plated in duplicate onto a pyrogen-free 96 well microplate (Associate Cape Cod; Falmouth, Mass., USA). After incubation at 37° C. for 10 minutes, 50 μl of reconstituted ENDOCHROME™ LAL reagent was added per well. The plate was incubated at 37° C. for another 10 minutes followed by addition of 100 μl of S-2423 Substrate-Buffer solution (1:1 dilution) per well. Next, the plate was set at room temperature for 3 minutes until differential coloration was visible in the endotoxin standard wells. The reaction was stopped by adding 100 μl of 20% acetic acid. Absorbance at 410nm was measured using the MR5000 Plate Reader (Dynatech; Chantilly, Va., USA), and the linear range of the endotoxin standard curve was used to determine endotoxin concentrations. All sample dilutions and additions of lysate to the microplate wells were made using pyrogen-free tips and in a biosafety hood.

### Example 6

### MEP HYPERCEL™ Binding Capacity and Protein Recovery

The BioSepra MEP HYPERCEL™ sorbent is optimized for capture and purification of monoclonal and polyclonal IgG. Binding capacities of more than 30 mg IgG per ml of sorbent, at 10% breakthrough, have been reported for human polyclonal IgG and murine monoclonal IgG₁ (Boschetti et al, Genetic Engineering News, 20 (13):1-4 (2000)). In our experiments, pure solutions of two monoclonal antibodies containing residual levels of contaminating endotoxin were applied to MEP HYPERCEL™ column as described in materials and methods for small-scale purification. MEP HYPERCEL™ binding capacity for mAb1 and mAb2 was measured to be about 26 mg IgG per ml of sorbent, at 23% and 34% breakthrough respectively (Table 1). To utilize the advantage of mild acidic conditions (pH below 4.5) for antibody elution from MEP HYPERCEL™, a buffer with pH 4.0 was used. As noted at least for one antibody, mAb1, protein recovery was affected by the pH of the elution buffer. The protein recovery as a percentage ratio of antibody bound to the column was calculated and antibody eluted from the column.

When a buffer with pH 3.0 was applied after a buffer with pH 4.0, an additional small fraction of mAb1 was eluted. The results are shown in Table 1 and Table 2.

TABLE 1

| | mAb1 | mAb2 |
|---|---|---|
| Amount of antibody applied to the column (mg) | 215 | 250 |
| Amount of antibody in the flow through fraction (mg) | 48.4 | 84 |
| Amount of antibody eluted at pH 4.0 (mg) | 160.4 | 161.1 |
| Amount of antibody eluted at pH 3.0 (mg) | 2.2 | 0 |
| Dynamic binding capacity^{a} (mg/ml of gel) | 26.5 | 26.4 |
| Total protein recovery^{b} (%) | 98 | 97 |
| Endotoxin concentration in the load (EU/mg) | 0.65 | 0.38 |
| Endotoxin concentration in the eluate (EU/mg) | <0.03 | <0.03 |
| Endotoxin removal efficiency^{c} (%) | 100 | 100 |

^{a}Dynamic binding capacity is measured with antibody concentration 2 mg/ml and at a flow rate 76 cm/h using 6.28 ml column (1 × 8 cm).
^{b}Protein recovery is expressed as a percentage ratio of antibody bound to the column and antibody eluted from the column.
^{c}Endotoxin removal efficiency is a percentage ratio of endotoxin concentration in the load and endotoxin concentration in the eluted antibody.

TABLE 2
Removal of Endotoxin Spiked into an Antibody Solution^{a}.

| | mAb1 | mAb2 |
|---|---|---|
| Amount of antibody applied to the column (mg) | 20 | 20 |
| Amount of antibody eluted at pH 4.0 (mg) | 16 | 19.8 |
| Amount of antibody eluted at pH 3.0 (mg) | 1.9 | 0 |
| Endotoxin concentration in the load (EU/mg) | 6.5 | 15 |
| Fraction of unbound endotoxin^{b} (%) | 57 | 23 |
| Endotoxin concentration in the eluate (EU/mg) | <0.03 | <0.03 |
| Endotoxin removal efficiency^{c} (%) | 100 | 100 |

^{a}Endotoxin from E. coli 055: B5 was spiked into antibody solution. Removal of endotoxin was performed on 6.28 ml MEP HYPERCEL ™ column (1 × 8 cm).
^{b}Fraction of unbound endotoxin is expressed as a percentage ratio of total endotoxin applied to the column and endotoxin detected in the flow through fraction and in the wash.
^{c}Endotoxin removal efficiency is expressed as in Table 1.

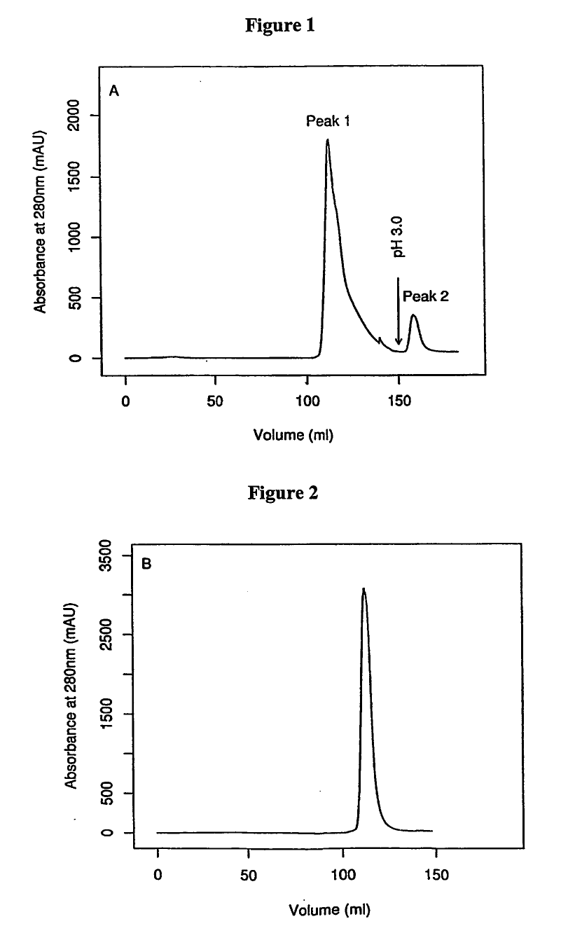

The recovery of mAb1 was 80% after elution with pH 4.0 and 90% after elution of the additional fraction at pH 3.0 (Table 2). The elution profiles are shown in FIGS. 1 and 2. These runs use a 1 cm inner diameter×8 cm bed height MEP HYPERCEL™ column. The flow rate was 76 cm/h. 20 mg of pure antibody solution spiked with stock endotoxin were loaded for each run. (A) Peak 1 represents mAb1 fraction eluted at pH 4.0, and peak 2 is mAb1 fraction eluted at pH 3.0. (B) Protein peak represents mAb2 fraction eluted at pH 4.0. It can be seen that elution of mAb1 at pH 4.0 (FIG. 1) resulted in broader protein peak and longer elution time than that of mAb2 (FIG. 2). This indicates a stronger interaction of mAb1 with the sorbent. On the basis of these observations, in subsequent experiments we routinely used elution buffer pH 3.0 for mAb1 and pH 4.0 for mAb2.

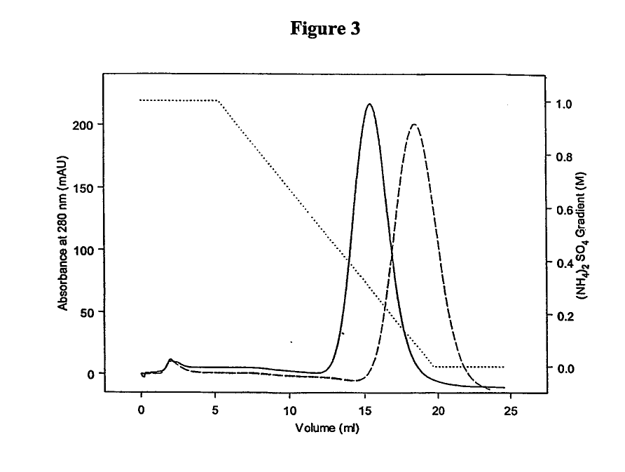

Binding of antibodies to MEP HYPERCEL™ is based on mild hydrophobic interactions and molecular recognition (Guerrier et al, In: Subramanian, G. (Ed.), European Conference on Antibodies Production and Purification, Paris, France, Oct. 27-29, 1-9 (1999)). To test if there may be a difference in the hydrophobic behavior of these two antibodies, we studied their interaction with Phenyl Sepharose 6 Fast Flow matrix. FIG. 3 shows that mAb1 was more hydrophobic and had a stronger binding to Phenyl Sepharose than mAb2. The flow rate was 1 ml/min. 1 mg of each antibody, containing 1 M ammonium sulfate, was loaded on the column. Antibody was eluted with negative gradient of 1-0 M ammonium sulfate. Absorbance of mAb1 is the dashed line, absorbance of mAb2 is the solid line, and ammonium sulfate gradient is the dotted line. Therefore, these two antibodies were different in regard to their interaction with hydrophobic matrixes.

Pure solutions of antibodies purified by Protein A affinity chromatography as described above were used in the experiments. The purity of these antibodies was more than 99% (data not shown), but they contained contaminating endotoxin. As presented in Table 1 and 2, we tested antibody solutions containing low native endotoxin as well as stock E. coli endotoxin spiked into antibody solutions. There was a possibility that endotoxin presented in antibody solutions could effect protein recovery.

It has been reported that the recovery of hemoglobin on Sterogene Anticlean Etox column was decreased due to the presence of endotoxin in protein solutions. Sterogene Anticlean Etox column has no affinity for hemoglobin. Nevertheless, when endotoxin was present in the protein solution and formed complexes with hemoglobin the binding of endotoxin to the column kept the protein in the column as well. In our experiments antibody were bound to MEP HYPERCEL™ column as well as a fraction of endotoxin. In case of antibody solutions spiked with endotoxin, 43% of total endotoxin in mAb1 and 77% in mAb2 solution were adsorbed by the column (Table 2). Therefore, besides different hydrophobic behavior of these two antibodies, we cannot exclude that possible interactions during the chromatographic process between antibody and endotoxin, antibody and matrix, and endotoxin and matrix, could affect protein recovery on MEP HYPERCEL™. Protein binding is apparently higher for more hydrophobic antibodies.

### Example 7

### Efficiency of Endotoxin Removal in Small-Scale Purification

The efficiency of endotoxin removal was calculated as a percentage ratio of endotoxin concentration in the antibody solution applied to the column and endotoxin concentration in the eluted protein fraction. MEP HYPERCEL™ was exceptionally successful in removing endotoxin from antibody solutions containing low remaining native endotoxin (Table 1) as well as stock endotoxin from E.Coli spiked into antibody solution (Table 2). The removal efficiency of 100% means that endotoxin concentration in the final antibody preparations was below 0.03 EU/ml, the test limit of LAL chromogenic assay. Depending on the initial endotoxin concentration 1-3 log reduction in endotoxin was achieved.

To elucidate the mechanism of endotoxin removal, we passed PBS buffer spiked with E.Coli endotoxin through MEP HYPERCEL™ column to see if contaminating endotoxin binds to the gel in the absence of protein and if it can be eluted from the matrix by low pH buffer. 50 ml of PBS containing 66.8 EU/ml of endotoxin was applied to the column. The chromatographic process was performed as described in materials and methods for small-scale purification. 55% of the total endotoxin was bound to the column, while 45% was detected in the flow through fraction and wash. Elution buffer (pH 3.0) did not elute endotoxin from the column. This suggested that endotoxin interacts with the resin. It is possible that this interaction occurs via hydrophobic part of endotoxin, lipid A component The fraction of endotoxin that did not bind to the column may have contained endotoxin of a different chemical structure and physical characteristics. It has been reported that endotoxin aggregates create steric restrictions for its interaction with sorbents and decrease endotoxin absorption (Petsch and Anspach, J. Biotechnol. 76, 97-119 (2000)).

### Example 8

### Efficiency of Endotoxin Removal in Scaled-Up Purification

Based on the small-scale purification experiments, a protocol for endotoxin removal from antibody solutions on a gram-scale was defined (see materials and methods). It is always desirable in an endotoxin removal step to have both high endotoxin removal efficiency and high protein recovery. Small-scale purification was extremely efficient in endotoxin removal and antibody recovery (Table 1). To investigate the efficiency of endotoxin removal and protein recovery during repeated scaled up purification, we performed three consecutive runs with mAb1 fractions containing different initial concentrations of native endotoxin. The results are shown in Table 3.

TABLE 3

| | Run 1 | Run 2 | Run 3 |
|---|---|---|---|
| Amount of antibody applied to | 400 | 840 | 1575 |
| the column (mg) | | | |
| Protein recovery (%) | 86 | 85 | 77 |
| Endotoxin concentration in | 50 | 32 | 25 |
| the load (EU/mg) | | | |
| Total endotoxin applied to the | 20000 | 26880 | 40478 |
| column (EU) | | | |
| Total endotoxin in the flow thru | 8800 | 11000 | ND^{b} |
| and wash (EU) | | | |
| Fraction of unbound endotoxin (%) | 44 | 41 | ND |
| Endotoxin concentration in | 6.8 | 6.3 | 0.83 |
| the eluate (EU/mg) | | | |
| Endotoxin removal efficiency (%) | 86 | 80 | 96 |

^{a}Purification was performed on 250 ml column (5 × 13 cm) of MEP HYPERCEL ™. Protein recovery, fraction of unbound endotoxin and endotoxin removal efficiency are expressed as in Table 1 and Table 2.
^{b}ND—not determined.

The efficiency of endotoxin removal in runs 1 and 2 was lower than in small-scale experiments and final antibody preparations had higher endotoxin contamination. About 12% and 17% of total endotoxin applied to the column was recovered with the antibody, while 44% and 42% of endotoxin were absorbed by the column. We also noted an increase in the backpressure and reduction in the flow rate of the column after these runs. We suggested that endotoxin absorbed to the column created a problem of efficient column regeneration. Apparently, a wash with 1N sodium hydroxide for 1 h that was routinely done in a small-scale purification and before runs 1 and 2 was not able to remove all endotoxin bound to MEP HYPERCEL™. When the column is not properly cleaned, leaking endotoxin could contaminate antibody fraction or a column binding capacity for endotoxin could be decreased. To regenerate the column and restore its flow characteristics before run 3 we used 50% ethylene glycol. This cleaning procedure was very efficient. As seen in Table 3, the level of endotoxin was significantly reduced in run 3.

Protein recovery in all three runs was acceptable but lower than in small-scale purification. Moreover, in run 3 the column had the highest endotoxin removal efficiency, 96%, but the lowest antibody recovery, 77%. Therefore, when optimizing the method of endotoxin removal with regard to the specific product a compromise may need to be made between high endotoxin removal efficiency and high protein recovery.

In the drawings and specification, there have been disclosed typical preferred embodiments of the invention and, although specific terms are employed, they are used in a generic and descriptive sense only and not for purposes of limitation, the scope of the invention being set forth in the following claims. Obviously many modifications and variations of the present invention are possible in light of the above teachings. It is therefore to be understood that within the scope of the appended claims the invention may be practiced otherwise than as specifically described.

What is claimed is:

1. A method for removing endotoxin contaminants from a protein solution, comprising:
adjusting the pH of a protein solution to a pH of from about 7.5 to about 10.5;
binding the protein to a hydrophobic charge induction chromatography sorbent; and
eluting the protein from the sorbent using an elution buffer having a pH of from about 2.5 to about 5.0.

2. The method of claim 1 wherein the protein is an antibody or fragment thereof.

3. The method of claim 2 wherein the antibody fragment is a human IgG Fc fragment.

4. The method of claim 1 wherein the hydrophobic charge induction chromatography comprises a cellulose matrix linked to a ligand selected from the group consisting of Mercapto-Ethyl- Pyridine (4-MEP) or its analogs.

5. The method of claim 1 wherein the hydrophobic charge induction chromatography comprises a cellulose matrix linked to a 4-Mercapto-Ethyl-Pyridine (4-MEP) analog selected from the group consisting of compounds having the structure 4-A-B-C, where A is an amino or hydroxyl group; B is a linear or branched hydrocarbon having from 1 to 8 carbon atoms, and C is pyridine.

6. The method of claim 1 wherein the hydrophobic charge induction chromatography comprises a cellulose matrix linked to 4-Mercapto-Ethyl-Pyridine (4-MEP) ligand.

7. The method of claim 1 further comprising pre-purifying the protein solution using affinity chromatography.

8. The method of claim 7 wherein the affinity chromatography is selected from the group consisting of Protein A Hydroxyapatite affinity chromatography and Prosep A affinity chromatography.

9. The method of claim 1 wherein the amount of endotoxin contaminant present in the protein solution after elution is less than 0.03 EU/ml.

10. The method of claim 1 further comprising recovering the eluted protein to produce a protein composition substantially free of endotoxin contaminants.

11. The method of claim 1 wherein the pH of the protein solution is adjusted to a pH of from about 8.0 to about 9.0.

12. The method of claim 1 wherein the protein is eluted using an elution buffer having a pH of from about 3.5 to about 4.5.

13. The method of claim 1 further comprising washing the protein bound to the hydrophobic charge induction chromatography sorbent using a buffer having a pH of from about 7.0 to about 7.5 prior to eluting the protein from the sorbent.

14. A protein composition produced according to the method of claim 9.

15. The protein composition of claim 14 having an endotoxin contaminant concentration equal to or less than 5 endotoxin units (EU) per dose per kilogram body weight when administered intravenously in a one hour period.

16. The protein composition of claim 14 wherein the protein is an antibody or fragment thereof.

17. The protein composition of claim 16 wherein the protein is a human IgG Fc fragment.

18. A method for removing endotoxin contaminants from a antibody or antibody fragment protein solution, comprising:
adjusting the pH of the antibody or antibody fragment protein solution to a pH of from about 8.0 to about 9.0;
binding the antibody or antibody fragment to a hydrophobic charge induction chromatography sorbent comprising a cellulose matrix linked to 4-Mercapto-Ethyl-Pyridine (4-MEP) ligand; and
eluting the antibody or antibody fragment from the sorbent using an elution buffer having a pH of from about 3.0 to about 4.5.

19. The method of claim 18 further comprising washing the antibody or antibody fragment bound to the hydrophobic charge induction chromatography sorbent with a buffer having a pH of from about 7.0 to about 7.5 prior to eluting the antibody or antibody fragment from the sorbent.

20. The method of claim 19 further comprising recovering the eluted protein to produce a protein composition substantially free of endotoxin contaminants.
